(19) United States
(12) Patent Application Publication
(10) Pub. No.: US 2009/0181026 A1
(43) Pub. Date: Jul. 16, 2009

(54) Tomoregulin-2-antibody compositions and methods for the diagnosis and treatment of Alzheimer's disease

(76) Inventors: Donald A. Siegel, Flushing, NY (US); May Huang, Whitestone, NY (US)

Correspondence Address: Davidson, Davidson & Kappel, LLC, 485 7th Avenue, 14th Floor, New York, NY, 10018, US

(21) Appl. No.: 12/157,182

(22) Filed: Jun. 6, 2008

Related U.S. Application Data
(60) Provisional application No. 60/933,395, filed on Jun. 6, 2007.

(51) Int. Cl. A61K 39/395; A61K 38/17; G01N 33/53; C07K 14/435; A61K 39/00; A61P 25/28

(52) U.S. Cl. 424/139.1; 514/12; 435/7.1; 530/326; 424/184.1

(57) ABSTRACT

The present invention is directed to methods which exploit the discovery of tomoregulin-2 (TR2) in amyloid plaques in Alzheimer's disease using TR2-sepcific antibodies; methods and compositions comprising TR2-specific antibodies for the diagnosis and treatment of Alzheimer's disease; methods and compositions comprising immunizing TR2 peptides to induce an immune response, thereby reducing/inhibiting plaque formation; and methods and compositions comprising enzyme inhibitors to blocking the enzymatic release of TR2 ectodomain from the cell surface or inhibit TR2 protein translation by RNA interference technology.

This application claims priority to U.S. Provisional Patent Application No. 60/933,395, filed Jun. 6, 2007, the disclosure of which is hereby incorporated by reference.

### BACKGROUND OF THE INVENTION

Neuritic plaques are one of the hallmarks of Alzheimer's disease (AD). They are extracellular deposits composed primarily of the protein β-amyloid (Aβ), a fragment of the larger molecule, amyloid precursor protein (APP) (Robakis N. K. (1994) Beta-amyloid and amyloid precursor protein: Chemistry, molecular biology and neuropathology, in Alzheimer's Disease, pp 317-333. Lippincott Raven, New York). Based on morphology, plaques are organized into distinct categories believed to represent a progression of plaque development occurring during the course of AD (Terry et al., (1994) Structural basis of the cognitive alterations in Alzheimer disease, in Alzheimer's Disease, pp 179-196. Lippincott Raven, New York; Yamaguchi et al., (1988) Acta Neuropathol (Berl) 76, 541-549). Diffuse plaques, the earliest formed, are composed of amorphous extracellular deposits of Aβ, and lack dystrophic neurites. These progress to primitive neuritic plaques composed of bundles of amyloid fibrils and dystrophic neurites, which mature to neuritic plaques that possess dense amyloid cores and dystrophic neurites. Hypermature plaques, dense amyloid deposits devoid of dystrophic neurites, represent the last stage in plaque development. Reactive astrocytes and microglia frequently surround plaques, and may contribute to plaque formation as the disease process (Terry et al., 1994).

One of the leading theories of Alzheimer's disease pathogenesis hypothesizes that increasing levels of Aβ is the initiating event in the disease (Hardy and Selkoe, (2002) Science 297, 353-356). Primary evidence in support of this theory is genetic. Mutations in the APP and presenilin 1 (PS1) genes (PS1 is a protein involved in APP processing to Aβ) are known to increase Aβ levels, and these mutations are linked to familial AD (Tanzi and Bertram, (2005) Cell 120, 545-555). This hypothesis is supported by animal and human studies. LaFerla and colleagues (Oddo et al., (2004) Neuron 43, 321-332), using a triple transgenic mouse model of AD, demonstrated that Aβ pathology precedes tau pathology; and Holtzman and colleagues (Brendza et al., (2005) J Clin Invest 115, 428-433) using another mouse model have shown that dystrophic neurites are secondary to Aβ deposition. In humans, a prospective clinical/pathology study by Corey-Bloom and colleagues (Tiraboschi et al., (2004) Neurology 62, 1984-1989), compared levels of neurofibrillary tangles and neuritic plaque load with severity of dementia in AD patients. While these authors confirmed earlier studies that showed a direct correlation between neurofibrillary tangles (intracellular accumulations of the microtubule associated protein tau) content and increasing disease severity, they also found that early in the disease process neurofibrillary tangles were rare, and that neuritic plaques appeared to be a more sensitive marker of AD.

Numerous studies in humans have correlated elevated levels of TGF-β with plaques formation in AD (Amara, F. M. et al. (1999) Brain Res Mol Brain Res 71, 42-49) (Burton, T. et al. (2002) Biochem Biophys Res Commun 295, 702-712). The source of TGF-β is believed to be part of the immune response mounted by microglia and astrocytes to initial deposits of amyloid or even to trauma. It is believed that part of the mechanism through which TGF-β contributes to amyloid plaque formation is through increased local synthesis of APP and β-amyloid from astrocytes.

TR2 is a glycosylated, single pass, type I, transmembrane protein predominately expressed in brain. It possesses a unique extracellular domain, containing both an epidermal growth factor-like motif and two follistatin-like modules (Horie et al. (2002) Genomics 67,146-152; Uchida et al., (1999) Biochem Biophys Res Commun 266, 593-602). Follistatin is an inhibitor in the TGF-β signal transduction pathway. The extracellular domain (ECD) can be cleaved by cell surface proteases, (Horie et al., 2002; Lin et al., (2003) Life Sci 73, 1617-1627; Uchida et al., 1999), releasing it into the extracellular space. Cleavage is stimulated by proinflammatory cytokines interleukin 1β (IL1β) and tumor necrosis factor a (TNFα) (Lin et al., 2003). The TR2 extracellular domain has been shown to function as neurotrophic factor for hippocampal and mesencephalic dopaminergic neurons, and appears to increase dendrite length in the latter cell type (Horie et al., 2002).

### SUMMARY OF THE INVENTION

In certain embodiments, the present invention is directed to methods for the identification of tomoregulin-2 (TR2) in amyloid plaques in Alzheimer's disease by TR2-specific antibodies. In other embodiments, the present invention is directed to methods and compositions comprising TR2-specific antibodies for the diagnosis and treatment of Alzheimer's disease. In other embodiments, the present invention is directed to methods and compositions comprising inducing an immune response to TR2 or fragments thereof, thereby reducing/inhibiting plaque formation. In yet other embodiments, the present invention is directed to methods and compositions comprising enzyme inhibitors to block the enzymatic release of TR2 ectodomain from the cell surface.

In certain embodiments, the present invention is directed to methods for the treatment of protein folding disorders, including but not limited to Alzheimer's disease, comprising administering a composition to a human patient in need thereof, said composition comprising a protein having an amino acid sequence that corresponds to a portion of the extracellular domain of TR-2 and inhibits cleavage of a portion of the extracellular domain of tomoregulin-2 (TR-2) by binding to a TR-2 protease, thereby preventing the protease from binding and cleaving the extracellular domain of TR-2.

In other embodiments, the present invention is directed to methods for the treatment of protein folding disorders, including but not limited to Alzheimer's disease, comprising administering an antibody to a human patient in need thereof, wherein said antibody is specific for a portion of the extracellular domain of TR-2 and inhibits cleavage of a portion of the extracellular domain of TR-2 by binding to the extracellular domain of TR-2, thereby blocking a cleaving agent from cleaving the extracellular domain of TR-2.

In yet other embodiments, the present invention is directed to methods for inhibiting formation of plaques associated with Alzheimer's disease that comprise beta-amyloid and a portion of TR-2 in a human patient comprising administering to the human patient an effective amount an antibody specific for a portion of the extracellular domain of TR-2, wherein the antibody binds to a portion of the extracellular domain of TR-2, thereby preventing beta amyloid from binding to a portion of the extracellular domain of TR-2 to form plaques.

In other embodiments, the present invention is directed to method for removing plaques associated with Alzheimer's disease that comprise beta-amyloid and a portion of TR-2 in a human patient comprising administering to the human patient an effective amount an antibody specific for a portion of the extracellular domain of TR-2, wherein the antibody binds to a portion of the extracellular domain of TR-2, thereby initiating an immune response and removal of the plaques.

In other embodiments, the present invention is directed to methods for diagnosing protein folding disorders, including but not limited to Alzheimer's disease, in a human patient comprising: a) taking a sample of tissue or fluid from the human patient; b) adding an antibody specific for a portion of TR-2 to the sample of tissue or fluid to created a mixture; c) testing for binding of the antibody to a portion of TR-2; wherein detection of bound antibody is indicative of Alzheimer's disease.

In other embodiments, the present invention is directed to methods for monitoring the progression of protein folding disorders, including but not limited to Alzheimer's disease, comprising: a) taking a first sample of tissue or fluid from a human patient; b) adding an antibody specific for a portion of TR-2 to said first sample; c) testing for binding of the antibody to TR-2 and quantifying the amount of TR-2 present in said first sample; d) taking a second sample of tissue or fluid from the human patient at a point in time after the taking of the first sample; e) adding the antibody specific for a portion of TR-2 to said second sample; f) testing for binding of the antibody to TR-2 and quantifying the amount of TR-2 in said second sample; g) comparing the amount of TR-2 present in the first and second samples; wherein a difference in the amount of TR-2 present in said first and second samples is indicative of disease progression or regression.

In other embodiments, the present invention is directed to an immunogenic composition for the treatment and prevention of a protein folding disorder, including but not limited to Alzheimer's disease, comprising an effective amount of an antibody specific for a portion of TR-2.

In yet other embodiments, the present invention is directed to an immunogenic composition comprising an effective amount of an isolated protein fragment having an amino acid sequence that corresponds to a portion of the extracellular domain of TR-2.

In other embodiments, the present invention is directed to a vaccine effective in treating a protein folding disorder, including but not limited to Alzheimer's disease, in a human subject comprising an isolated protein fragment having the amino acid sequence set forth in SEQ ID NO:1 and a pharmaceutically acceptable carrier.

In other embodiments, the present invention is directed to a method for inhibiting cleavage of a portion of the extracellular domain of tomoregulin-2 (TR-2) in vitro for the study of protein folding disorders, including but not limited to Alzheimer's disease, comprising contacting a composition comprising a protein having an amino acid sequence that corresponds to a portion of the extracellular domain of TR-2 to a tissue sample comprising a TR-2 protease, allowing the protein to bind to the TR-2 protease, thereby preventing the protease from binding and cleaving the extracellular domain of TR-2.

In other embodiments, the present invention is directed to a method for inhibiting cleavage of a portion of the extracellular domain of TR-2 in vitro for the study of protein folding disorders, including but not limited to Alzheimer's disease, comprising contacting an antibody specific for a portion of the extracellular domain of TR-2 to a tissue sample comprising TR-2, allowing the antibody to bind to the extracellular domain of TR-2, thereby blocking a cleaving agent from cleaving the extracellular domain of TR-2.

In other embodiments, the present invention is directed to a method of treating a protein folding disorder, including but not limited to Alzheimer's disease, comprising administering an effective amount of a composition to a human patient in need thereof, said composition comprising an inhibitor compound that prevents TR-2 release by inhibiting the enzyme(s) that cleave TR-2 from the cell surface, and a TR-2 antibody.

In yet other embodiments, the present invention is directed to a method of treating a protein folding disorder, including but not limited to Alzheimer's disease, in a human patient, comprising administering an effective amount of a TR-2 antibody to a human patient, and using RNA interference technology to prevent the cellular expression of TR-2.

In other embodiments, the present invention is directed to a method of treating a protein folding disorder, including but not limited to Alzheimer's disease, comprising administering to a human patient an inhibitor compound that prevents TR-2 release by inhibiting the enzyme(s) that cleave TR-2 from the cell surface; administering to said patient a compound that prevents cleaved TR-2 from interacting with beta-amyloid; and using RNA interference technology to prevent the cellular expression of TR-2.

In other embodiments, the present invention is directed to a composition for the treatment of protein folding disorders, including but not limited to Alzheimer's disease, comprising, said composition comprising: an inhibitor compound that prevents TR-2 release by inhibiting the enzyme(s) that cleave TR-2 from the cell surface; and a TR-2 antibody.

Other embodiments and advantages of the present invention will become apparent from the disclosure herein.

### BRIEF DESCRIPTION OF THE DRAWINGS

FIG. 1A is a schematic representation of TR2. N=N-glycosylation sites, O=O-glycosylation site. Arrows identify canonical protease cleavage sites. TR2-Ab recognition site is indicated. FIG. 1B is a Western blot of recombinant TR2 and TR2-ECD from stably transformed CHO cells, detected with TR2 Ab. Lanes: 1=Full length TR2, 2=TR2-ECD, 3=Vector control, 4=Untransfecteded cells, 5=Adult mouse brain, 6=PO mouse brain. Molecular wt. stds. indicated on right.

FIGS. 2A-2H depict TR2 and Aβ Immunoreactivity (IR) in normal and Alzheimer cortex. Scale bar=500μ.

FIG. 3 depicts TR2 and Aβ immunocytochemistry on human AD cortex. A and D diffuse deposits, B and E neuritic plaques without dense cores, C and F dense core plaques. In all cases, morphologies of TR2 and Aβ plaques are indistinguishable. Scale bar=10μ.

FIG. 4 depicts a double immunofluorescent confocal microscopy of human AD plaques stained for TR2 and Aβ. Arrows identify double stained plaques; arrowheads Aβ only plaques. Scale bar=100μ. Rows B shows the same plaque stained for TR2. Row C shows a series of z-planes through the plaque boxed in Row B. Sections begin at the bottom of the plaque (left panel) and continue up through the top (right panel). Scale bar=10μ.

FIG. 5 depicts TR2 and Aμ immunocytochemistry on frontal cortex of a 12-month-old PS1/APP transgenic mouse. A) TR2 staining: arrows point to examples of plaque “ghosts”. B) Aβ 6E10 antibody staining: arrows point to Aβ plaques. C) TR2-immunoreactivity, enlargement from A. Arrowheads identify “ghost” plaques with surrounding process-like staining. Arrows identify neuropil thread-like structures. Scale bar=50μ.

FIG. 6 depicts A) TR2 neuronal staining in Alzheimer's disease cortex. TR2-IR is observed in the soma and proximal processes of two cortical pyramidal cells. In the neuron on the right staining is seen for some distance along in the apical dendrite (arrowhead). Similar staining is observed in the control tissue. Scale bar=50μ. B) Western analysis of TR2 and paired helical filament tau on normal human and AD brain samples. Three samples of normal aged brain (N) and four samples of AD brain (three severe (AD) and one moderate (M)) were evaluated with antibodies to TR2 (a) and phosphorylated paired helical filament tau (CP13 antibody) (b). 10 μg of protein were loaded per lane. Lanes 8 in both a and b blots are normal mouse brain used as a positive controls. MW markers at right, kD.

FIG. 7 depicts the TR2-TGFβ futile cycle. TGFβ stimulates Aβ production; full-length TR2 may function by blocking TGFβ signaling. Cleavage of the TR2 extracellular domain, stimulated by IL-β1 and TNFα released from activated astrocytes and microglia, would prevent TR2 from inhibiting TGFβ signaling and simultaneously make the TR2-ECD available to interact with Aβ and form amyloid deposits. This in turn, would cause the activation of more astrocytes and microglia, the release of additional IL-β1, TNFα and TGF-β and renew the cycle.

FIG. 8 depicts the TR2/TGFβ Signal Transduction Pathway.

### DETAILED DESCRIPTION OF THE PREFERRED EMBODIMENTS

### Definitions

The term “antibody,” as used herein, refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody multimers and antibody fragments as well as variants (including derivatives) of antibodies, antibody multimers and antibody fragments. Examples of molecules which are described by the term “antibody” herein include, but are not limited to: single chain Fvs (scFvs), Fab fragments, Fab′ fragments, F(ab′)₂, disulfide linked Fvs (sdFvs), Fvs, and fragments comprising or alternatively consisting of, either a VL or a VH domain. The term “single chain Fv” or “scFv” as used herein refers to a polypeptide comprising a VL domain of antibody linked to a VH domain of an antibody.

Antibodies of the invention include, but are not limited to, monoclonal, multispecific, human or chimeric antibodies, single chain antibodies, Fab fragments, F(ab′) fragments, anti-idiotypic (anti-Id) antibodies (including, e.g., anti-Id antibodies to antibodies of the invention), intracellularly-made antibodies (i.e., intrabodies), and epitope-binding fragments of any of the above. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

Antibodies of the invention may also include multimeric forms of antibodies. For example, antibodies of the invention may take the form of antibody dimers, trimers, or higher-order multimers of monomeric immunoglobulin molecules. Dimers of whole immunoglobulin molecules or of F(ab′)₂ fragments are tetravalent, whereas dimers of Fab fragments or scFv molecules are bivalent. Individual monomers without an antibody multimer may be identical or different, i.e., they may be heteromeric or homomeric antibody multimers. For example, individual antibodies within a multimer may have the same or different binding specificities.

Multimerization of antibodies may be accomplished through natural aggregation of antibodies or through chemical or recombinant linking techniques known in the art.

Unless otherwise defined in the specification, specific binding or immunospecific binding by an anti-TR2 antibody means that the anti-TR2 antibody binds TR2, or a fragment thereof, but does not significantly bind to (i.e., cross react with) proteins other than TR2. An antibody that binds TR2 protein and does not cross-react with other proteins is not necessarily an antibody that does not bind said other proteins in all conditions; rather, the TR2-specific antibody of the invention preferentially binds TR2 compared to its ability to bind said other proteins such that it will be suitable for use in at least one type of assay or treatment, i.e., give low background levels or result in no unreasonable adverse effects in treatment. It is well known that the portion of a protein bound by an antibody is known as the epitope. An epitope may either be linear (i.e., comprised of sequential amino acids residues in a protein sequences) or conformational (i.e., comprised of one or more amino acid residues that are not contiguous in the primary structure of the protein but that are brought together by the secondary, tertiary or quaternary structure of a protein). Given that TR2-specific antibodies bind to epitopes of TR2, an antibody that specifically binds TR2 may or may not bind fragments of TR2 and/or variants of TR2 (e.g., proteins that are at least 90% identical to TR2) depending on the presence or absence of the epitope bound by a given TR2-specific antibody in the TR2 fragment or variant. Likewise, TR2-specific antibodies of the invention may bind species orthologues of TR2 (including fragments thereof) depending on the presence or absence of the epitope recognized by the antibody in the orthologue. Additionally, TR2-specific antibodies of the invention may bind modified forms of TR2, for example, TR2 fusion proteins. In such a case when antibodies of the invention bind TR2 fusion proteins, the antibody must make binding contact with the TR2 moiety of the fusion protein in order for the binding to be specific. Antibodies that specifically bind to TR2 can be identified, for example, by immunoassays or other techniques known to those of skill in the art.

The term “derivative” as used herein, refers to a variant polypeptide of the invention that comprises, or alternatively consists of, an amino acid sequence of a TR2 polypeptide, a fragment of a TR2 polypeptide, or an antibody of the invention that immunospecifically binds to a TR2 polypeptide, which has been altered by the introduction of amino acid residue substitutions, deletions or additions. The term “derivative” as used herein also refers to a TR2 polypeptide, a fragment of a TR2 polypeptide, an antibody that immunospecifically binds to a TR2 polypeptide which has been modified, e.g., by the covalent attachment of any type of molecule to the polypeptide. For example, but not by way of limitation, a TR2 polypeptide, a fragment of a TR2 polypeptide, or an anti-TR2 antibody, may be modified, e.g., by glycosylation, acetylation, pegylation, phosphorylation, amidation, derivatization by known protecting/blocking groups, proteolytic cleavage, linkage to a cellular ligand or other protein, etc. A derivative of a TR2 polypeptide, a fragment of a TR2 polypeptide, or an anti-TR2 antibody, may be modified by chemical modifications using techniques known to those of skill in the art, including, but not limited to, specific chemical cleavage, acetylation, formylation, metabolic synthesis in the presence of tunicamycin, etc. Further, a derivative of a TR2 polypeptide, a fragment of a TR2 polypeptide, or an anti-TR2 antibody, may contain one or more non-classical amino acids. A polypeptide derivative possesses a similar or identical function as a TR2 polypeptide, a fragment of a TR2 polypeptide, or an anti-TR2 antibody, described herein.

The term “epitopes” as used herein refers to portions of TR2 having antigenic or immunogenic activity in an animal, preferably a mammal. An epitope having immunogenic activity is a portion of TR2 that elicits an antibody response in an animal. An epitope having antigenic activity is a portion of TR2 to which an antibody immunospecifically binds as determined by any method known in the art. Antigenic epitopes need not necessarily be immunogenic.

The term “fragment” as used herein refers to a polypeptide comprising an amino acid sequence of at least 5 amino acid residues, at least 10 amino acid residues, at least 15 amino acid residues, at least 20 amino acid residues, at least 25 amino acid residues, at least 30 amino acid residues, at least 35 amino acid residues, at least 40 amino acid residues, at least 45 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues, of the amino acid sequence of TR2, or an anti-TR2 antibody (including molecules such as scFv's, that comprise, or alternatively consist of, antibody fragments or variants thereof).

The term “fusion protein” as used herein refers to a polypeptide that comprises, or alternatively consists of, an amino acid sequence of an anti-TR2 antibody of the invention and an amino acid sequence of a heterologous polypeptide (i.e., a polypeptide unrelated to an antibody or antibody domain).

Antibodies of the present invention are preferably provided in an isolated form, and preferably are substantially purified. By “isolated” is intended an antibody removed from its native environment. Thus, for example, a polypeptide produced and/or contained within a recombinant host cell is considered isolated for purposes of the present invention.

The term “treatment” as used herein encompasses the slowing of the progression of a disease state.

“Protein folding disorders”, as used herein include any neurodegenerative condition such as, e.g, Alzheimer's disease, dementia, Huntington's disease, Parkinson's disease and prion-based spongiform encephalopathy (e.g., Creutzfeldt-Jakob disease).

Using antibodies directed against the TR2 sequence:

DDRENDLFLCDTNTCKFDGE (Seq. ID No. 1) (Siegel et al. (2006), J. Neurochem. 98:34-44; the disclosure of this reference, and all other references cited herein, is hereby incorporated by reference in their entireties for all purposes), TR2 has been shown to be closely associated with beta-amyloid (A-Beta) in amyloid plaques of Alzheimer's disease. Consequently antibodies, in whole or in part, or conjugated to other chemical moieties, such as, but not limited to fluorescent markers, could be used to diagnose Alzheimer's from bodily tissues or fluids, or treat Alzheimer's disease. Other antibodies directed against part of the TR2 antigenic sequence (DDRENDLFLCDTNTCKFDGE) (SEQ. ID No. 1), or other parts of the TR2 extracellular domain from amino acids 1 through 318 could also be used for detection or therapy of Alzheimer's disease. Such antibodies include those described by Uchida et al. (1999) Biochem. Biophys. res. Comm. 266:593-602: monoclonal antibody 2H8 (generated against the Glutathione-S-transferase-TR2 extracellular domain fusion protein made from amino acids 17-336) and also those described in US Patent Publications US2006/0292074A1 (antibody AT-19 scFv) and US2004/0096392A1 (cell line expressing antibody TMEFF#19 ATCC# PTA-4127), the disclosures of which are hereby incorporated by reference in their entireties.

The present invention includes antibodies that recognize TR2 and the extracellular domain of TR2 near the amino terminal of the protein, i.e., in the most distal follistatin module.

TR2, and more specifically portions of the TR2 extracellular domain, was found to be present in amyloid plaques found in Alzheimer's disease. Using double-immunofluorescent confocal microscopy (Siegel et al. 2006), i.e., using one antibody directed against beta amyloid (the predominant protein in Alzheimer amyloid plaques) and the TR2 antibody of the present invention, TR2 was found to be present throughout amyloid plaques and closely associated with beta-amyloid.

Thus, the present invention is directed to TR2 and its interaction with beta-amyloid and the finding that deposition of beta-amyloid in Alzheimer's disease may be increased by its binding to TR2. Therefore, embodiments of the invention are directed to blocking the interaction of TR2 with beta-amyloid, and slowing, inhibiting and/or monitoring the progression of Alzheimer's disease.

Thus, embodiments of the invention include immuno-therapies for Alzheimer's disease based upon the interaction of TR2 and beta amyloid. Certain preferred embodiments of these immuno-therapeutic methods involve using the TR2-ECD (including, but not limited to the 20 amino acid sequence used to make the TR2 antibody of the present invention) to inoculate a patient to cause their immune system to make antibodies to TR2-ECD. These antibodies may bind to TR2 already in plaques and cause the plaques to be removed by microglia/macrophages in the brain. This immunotherapy would also prevent TR2 from interacting with beta-amyloid in the first stage, consequently preventing TR2/beta-amyloid plaque formation altogether. See, e.g., U.S. Pat. Nos. 6,743,427 and 6,761,888, which discuss passive immunization therapy, and Buttini M. et al., J Neurosci. 25(40):9096-10101, (2005); Maier M, et al., J Neurosci. 26(18):4717-4728, (2006); Sigurdsson E M et al., J Neurosci. (2004 Jul. 14) ;24(28):6277-82; and Wilcock D M et al., J Neuroinflammation. 1(1):24. (2004), which discuss active and passive immunotherapy using beta-amyloid or beta-amyloid antibodies respectively.

Other embodiments of this invention include methods for blocking the cleavage or release of the TR2-ECD from the full-length transmembrane TR2 protein. Thus, in certain preferred embodiments of the present invention, AD may be treated by blocking release of the TR2-ECD from the cell surface. This may be done by (1) blocking the proteases that cleave TR2-ECD from the holoprotein or (2) blocking interleukin-1-beta (IL-1β) and/or tumor necrosis factor-alpha (TNF-α) from stimulating protease activity. If the release of TR2-ECD is blocked, it may be less likely to interact with beta-amyloid and form plaques. Lin et al. Life Sciences 73:1617-1627 (2003) state that TR2-ECD cleavage is partially blocked by tumor necrosis factor-alpha (TNF-α) protease inhibitors 1 & 2 (TAPI-1 & TAPI-2) and conclude that metalloproteases are at least partially responsible for TR2-ECD cleavage. Other metalloprotease inhibitors include, e.g., Amastatin-HCl, Arphamenine A, Arphamenine B, Bestatin-HCl, Diprotin A, EDTA-Na2, EGTA and Phosphoramidon.

Naturally occurring inhibitors of matrix metalloproteases include alpha 2-macroglobulin and other tissue inhibitors of metalloproteases (TIMPs) (S. Wojtowicz-Praga, Clinical Potential of Matrix Metalloprotease Inhibitors, Drugs R D 1(2):117-129 (1999))

IL-1β and TNFα are proinflammatory cytokines. They are released when there is inflammation. It has been shown that the initial formation of beta-amyloid plaques can stimulate an inflammatory response in the brain. This would cause the release of IL-1β and TNFα, which in turn would activate proteases, which in turn would cleave the TR2-ECD, which could interact with beta-amyloid to form plaques and stimulate/perpetuate and.immune response starting the cycle over.

In certain embodiments of the present invention, AD is treated through the use of RNAi technology with the TR2 antibodies of the present invention to block specific protein synthesis. This technique uses small interfering RNAs (siRNA), or short double-stranded RNA hairpins (shRNA) to block specific gene translation. Alternatively, plasmid or viral vectors carrying DNA versions of interfering RNAs may be used. A variety of delivery systems can be used to direct the interfering RNAs or DNAs to their target cells by direct injection into the brain, injection into the cerebral spinal fluid, or systemically by crossing the blood brain barrier (BBB) (e.g. by exploiting “the transport systems, enzymes or receptors that control penetration” 1 of the blood brain barrier or by endocytosis or transcytosis of the BBB, or by manipulating the BBB by osmotic or chemical means. These delivery systems include viral vectors (RNA, e.g. adenovirus, adenovirus associated virus and lentivirus), or incorporating the siRNA/shRNA or DNA into lipid or polymer coats (e.g. liposomes, cationic polymers) with or without specific antibodies or ligands in the coat to direct them to specific cell types, or by direct injection into the brain. (See, e.g., Raoul, C., et al., Nature Medicine 11(4):423-428, (2005); Ralph, G. S., et al. Nature Medicine 11(4):429-433, (2005); Soutschek, J., et al., Nature 432:173-178 (2004); and Xia et al., Nature Medicine 10(8):816-820, (2004)).

In certain embodiments of the present invention, the treatment of AD is administered via directed introduction of existing TR2 antibody into the brain. After the plaques are removed by macrophage/microglia, they will not do further damage.

In certain other embodiments of the present invention, the use of a small fragment of TR2-ECD may be used to treat AD by potentially blocking the TR2-ECD from binding beta-amyloid. In other embodiments, natural or synthetic molecules may be used to block the TR2-ECD from binding beta-amyloid.

Preferred embodiments of the present invention include using TR2 antibodies to detect TR2 levels in cerebral spinal fluid (CSF) or blood in AD patients to determine if someone has AD based on detection of TR2 levels. In more preferred embodiments, the presence of AD in patients may be determined by: a) taking a sample of tissue or fluid from a subject; b) adding an antibody specific for a portion of TR-2 to the sample of tissue or fluid to created a mixture; and c) testing for binding of the antibody to a portion of TR-2, wherein the detection of bound antibody is indicative of Alzheimer's disease.

In other embodiments of the present invention, TR2 antibody is used as a research tool to detect TR2/beta-amyloid plaques postmortem.

In certain embodiments of the present invention, the TR2 antibody, or segments thereof, may be used alone or in combination with other reagents (e.g. liposomes) for treatment of AD via systemic infusion or direct injection into the brain.

In certain embodiments of the present invention, the cDNA or genomic sequence of TR2 is used to identify specific mutation(s) in the gene that occur in Alzheimer's disease. For example by examining the TR2 gene in people with AD and comparing it to non-AD age matched people to identify specific mutation that can lead to AD.

The present invention also relates to pharmaceutical compositions which may comprise TR2 polynucleotides, TR2 polypeptides, antibodies, agonists, antagonists, or inhibitors, alone or in combination with at least one other agent, such as stabilizing compound, which may be administered in any sterile, biocompatible pharmaceutical carrier, including, but not limited to, saline, buffered saline, dextrose, and water. Any of these molecules can be administered to a patient alone, or in combination with other agents, drugs or hormones, in pharmaceutical compositions where it is mixed with excipient(s) or pharmaceutically acceptable carriers. In one embodiment of the present invention, the pharmaceutically acceptable carrier is pharmaceutically inert.

The present invention also relates to the administration of pharmaceutical compositions. Such administration is accomplished orally or parenterally. Methods of parenteral delivery include topical, intra-arterial, intramuscular, subcutaneous, intramedullary, intrathecal, intraventricular, intravenous, intraperitoneal, or intranasal administration. In addition to the active ingredients, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Ed. Maack Publishing Co, Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

For topical or nasal administration, penetrants appropriate to the particular barrier to be permeated are used in the formulation. Such penetrants are generally known in the art.

In certain embodiments of the present invention, antiinflammatories may be used to reduce the release of TR2-ECD in combination with the methods and compositions described herein. Antiinflammatories for use in the present invention include, but are not limited to, steroids, aspirin, amoxiprin, benorilate, choline magnesium salicylate, diflunisal, faislamine, methyl salicylate, magnesium salicylate, salicyl salicylate (salsalate), diclofenac, aceclofenac, acemetacin, bromfenac, etodolac, indometacin, ketorolac, nabumetone, sulindac, tolmetin, ibuprofen, carprofen, fenbufen, fenoprofen, flurbiprofen, ketoprofen, loxoprofen, naproxen, tiaprofenic acid, suprofen, mefenamic acid, meclofenamic acid, phenylbutazone, azapropazone, metamizole, oxyphenbutazone, sulfinprazone, piroxicam, lornoxicam, meloxicam, tenoxicam, celecoxib, etoricoxib, lumiracoxib, parecoxib, rofecoxib valdecoxib, nimesulide, licofelone and omega-3 fatty acids.

The invention has been described with reference to specific exemplary embodiments and examples thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense. Any reference to any patent or publication made herein is hereby incorporated by reference in its entirety for all purposes.

### EXAMPLES

### Example 1

### Materials:

Full-length mouse TR2 cDNA was obtained by screening a mouse brain cDNA library (Siegel et al., (2002) Int J Dev Neurosci 20, 373-389). Primer sequences used for PCR cloning of the TR2 full-length (TR2-FL) and ectodomain (TR2-ECD) cDNAs into the pIRES mammalian expression vector (Stratagene pIRES-hrGFP-1a) were as follows:

TR2-FL 5′ primer:

(SEQ ID No. 2)

5′- TFGAGCTCGCCACCATGGTGCTGTGGGAGTC-3′
TR2-FL 3′ primer:

(SEQ ID No. 3)

5′- CCTTGCGGCCGCCTAGATTAACCGTGTGGATG -3′
TR2-ECD 3′ primer:

(SEQ ID No. 4)

5′- TTGCGGCCGCCCGGACAGGGCCAGGAAC -5′

PCR conditions for amplifying the full length and extracellular domain of TR2 were: 94° C.×3 min., 56° C.×0.5 min., 72° C.×3 min.×30 cycles. DNA polymerase was Hot Start PFU Turbo from Stratagene. Restriction enzymes Not I and Sac I, T4 polynucleotide kinase, calf intestinal alkaline phosphatase, and T4 DNA ligase were from New England BioLabs, Inc. ATP and 100 mM dNTP set were from Invitrogen Corp. The eukaryotic resistance neomycin gene module pExchange EC-Neo was from Stratagene, as were XL10-Gold Ultracompetent Cells. Qiaquick PCR Purification Kit and Qiaex II Gel Extraction Kit were from Qiagen Inc. CHO-S cells, CD-CHO medium, H/T supplement (100×: 10 mM sodium hypoxanthine, 1.6 mM thymidine) and 200 mM L-glutamine supplement were from Gibco Corp., as was Lipofectamin 2000. Lysis buffer for CHO-S cells and normal mouse brains was described previously (Siegel et al., 2002).

Tissue Samples: Samples of human mid-temporal gyrus from 4 normal (2M, 2F) and 8 Alzheimer's disease cases (3M, 5F, all aged 75-85 years old) were obtained at autopsy; the mean delay between death and tissue removal was 11 hours (range 7-19 hours). All cases were evaluated by standard neuropathologic methods. Normal cases were free of any significant neuropathology (Braak stage 1-2). AD cases were characterized by the presence of numerous neurofibrillary tangles and neuritic plaques in the hippocampus and in the neocortex (Braak stage 5-6). Samples of human brain were homogenized in Tris-buffered saline pH 7.4 containing 10 mM sodium fluoride, 2 mM EGTA, 1 mM sodium vanadate and 1 mM PMSF. Immunohistochemical studies were performed on 50μ vibratome sections of neocortex. Tissues were fixed in formalin for up to 3 months prior to sectioning. PS1/APP mice (12 months old, male) were a gift from Dr Karen Duff. Brains were removed and fixed overnight at 4° C. in 4% paraformaldehyde, prior to being sectioned in the sagittal plane (50μ) on a vibratome.

Antibodies & Peptides. Rabbit affinity purified anti-TR2 antibody was prepared as previously described (Siegel et al., 2002). 6E10 mouse monoclonal (IgG1) antibody detects amino acid residues 1-17 of human Aβ peptide was from Signet Labs, Dedham, Mass. The 42 amino acid Aβ peptide was purchased from Bachem Bioscience, King of Prussia, Pa. Paired helical filament (PHF) antibody CP-13 is a mouse monoclonal IgG that specifically recognizes tau phosphorylated on serine 202 (Davies, 1999; Duff et al., 2000). Biotinylated goat anti-rabbit IgG, biotinylated goat anti-mouse IgG, avidin-peroxidase and Vectastain Elite ABC Peroxidase kit were from Vector Labs, Burlingame, Calif. Fluorescent goat anti-rabbit IgG (Alexa Fluor 488, A11034) and goat anti-mouse IgG (Alex Fluor 546, A11030) were from Molecular Probes.

### Methods:

Cloning of full length and extracellular domain of TR2 into a mammalian expression vector: The full length and extracellular domain of mouse TR2 cDNAs were cloned into the mammalian expression vector pIRES-hrGFP-1a by PCR of the original pBlueScript TR2 clone (Siegel et al., 2002). This vector uses a CMV promoter for gene expression and has an SV40 polyadenylation signal downstream to ensure proper 3′ mRNA processing. Primers for amplification of full length TR2 were prepared with a Sac I restriction site followed by a Kozak consensus sequence upstream of the 5′ start site, and a stop codon and Not I restriction site downstream of the 3′ end of TR2 (see materials for sequence). For cloning the TR2 extracellular domain, the same 5′ primer was used, but in conjunction with a 3′ primer containing sequence adjacent to the extracellular side of the transmembrane domain, followed by a stop codon and Not I restriction site. All primers had two or four base overhangs at their 5′ ends to improve restriction enzyme digestion. Following PCR, amplicons were purified by Qiagen spin column chromatography, digested with Sac I and Not I, and electrophoresed through a 1% low melt agarose gel. Bands of anticipated size were cut from the gel, purified with a Qiagen gel extraction kit, and cloned into the multiple cloning site of the pIRES vector. After transformation into E. coli (XL-1 Blues), plasmids were purified, sequences checked twice by sequencing, and the neomycin resistance gene (pExchange Module EC-Neo) inserted into each plasmid, as well as the empty pIRES vector, by Cre-mediated site specific recombination. The TR2-FL-Neo, TR2-ECD-Neo and pIRES-Neo control plasmids were again transformed into XL-1 Blues. The neo inserts were confirmed by restriction enzyme digestion and analysis.

Expression of the recombinant full length and extracellular domain of TR2: The full length and extracellular domain of TR2, along with the empty control plasmid, were transfected into CHO-S cells using Lipofectamine, and stably transfected cell lines selected using G418 sulfate following the Gibco CHO-S cell protocol. Cells were grown in CD-CHO medium supplemented with sodium hypoxanthine, thymidine and L-glutamine as described in CHO-S cell protocol. Cells from two T-75 flasks were spun at 100 g×10 min., washed once with phosphate buffered saline (PBS), spun a second time, and the pellet homogenized in ten volumes of cell lysis buffer (see materials). Lysate was spun at 20,000 g×15 min. at 4° C. and the supernatant saved. Protein was quantified by the Bradford method using bovine serum albumin, prepared in lysis buffer, as standard. Polyacrylamide gel electrophoresis (PAGE) and western analyses were performed as previously described (Siegel, et al., 2002).

Polyacrylamide Gel Electrophoresis and Western Analysis: Alzheimer and normal aged human tissues were homogenized as described above (Materials). Homogenates were frozen until used for analyses. Protein was quantified by the Bradford method. Ten micrograms of protein from each sample were loaded onto a 10% polyacrylamide gel. Duplicate PVDF membranes were prepared.

Immunohistochemistry and Immunofluorescence: Fifty-micron sections of normal aged human and Alzheimer's disease brains were used for both immunohistochemistry (IHC) and immunofluorescence (IF) experiments. Sections are pretreated with 70% formic acid for 15 min. (except where noted) prior to blocking in 1.5% normal goat serum (NGS), 1% bovine serum albumin (BSA), 0.05% Triton X-100 in PBS (blocking solution). IHC staining was done using TR2 Ab at 0.28 μg/ml, or 6E10 Aβ Ab at 0.2 μg/ml. Sections were blocked as above, then incubated with secondary antibodies. The secondary for TR2 was biotinylated goat anti-rabbit IgG used at 7.5 μg/ml in blocking solution, followed by avidin-peroxidase and DAB. The secondary for Aβ was goat anti-mouse used at 7.5 μg/ml in blocking solution, also followed by avidin—peroxidase and DAB. Vectastain Elite ABC Peroxidase Kit was used following manufacturer's instructions. For diluent, blocking solution was used.

For antibody-peptide competition experiments, TR2-Ab was preincubated with either its own 20 amino acid immunizing peptide at 0.13 μg/ml (55.3 nM), or with the 42 amino acid Aβ peptide at 0.25 μg/ml (55.4 nM). The Aβ Ab was blocked by preincubation with either the TR2 peptide or the A□ peptide as above. Blocked antibodies were used following the same procedures described above for normal IHC.

Immunofluorescence was done following a similar protocol. Brain sections are pretreated with 70% formic acid for 15 minutes, then blocked in 5% NGS and 0.2 % Triton-X100 in PBS. TR2 Ab was used at 4.4 ug/ml and 6E10 Aβ Ab at 2.5 ug/ml. The secondary antibody for TR2 was Alexa Fluor 488 goat anti-rabbit IgG at 1:500 dilution. The secondary for the 6E10 Aβ Ab was Alexa Flur 546 goat anti-mouse, IgG1 at a 1:2000 dilution. Diluent for both primary and secondary antibodies was 2% NGS and 0.1% Triton X-100 in PBS. Specimens were assessed using the Fluoview 500 scanning fluorescence confocal microscopy system built around an inverted BX61WI microscope (Olympus), employing Argon blue (488 nm em.) and green HeNe (543 nm em.) lasers along with 505-525 band pass and 560 long pass emission filters, respectively. Images of dual labeled specimens were collected in “sequential” mode, in which data is obtained using one laser and one detection channel at-a-time to avoid false co-localization resulting from signal cross-talk.

For the PS/APP transgenic mouse, brain sections were stained as described by Duffet al., 2000. For Aβ staining, 6E10 was used at 0.5 ug/ml, with goat anti-mouse IgG1-biotin at a 1:1000 dilution. Streptavin-HRP and diaminobenzidine were used for color development.

### Results

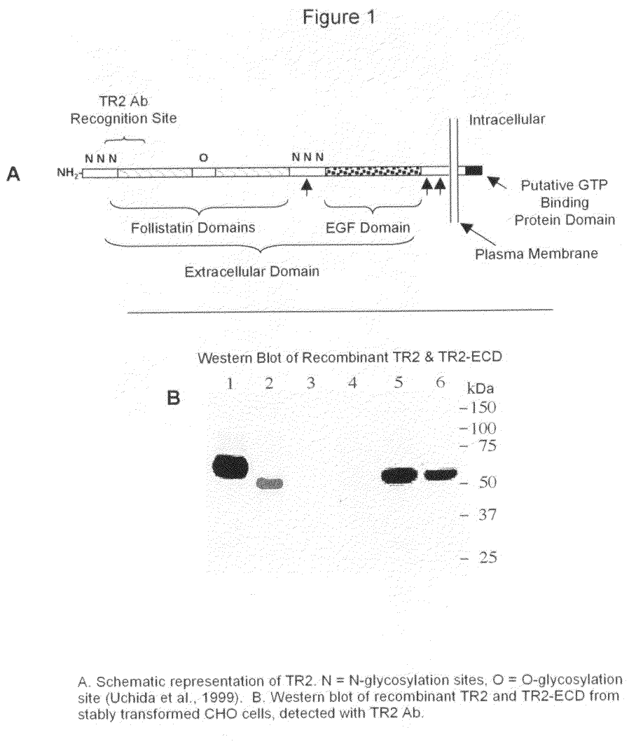

Characterization of TR2-Ab: Affinity purified rabbit anti-TR2 antibody (TR2-Ab) was raised against a twenty amino acid synthetic peptide located near the amino terminal end of the first (amino-most) follistatin domain of mouse TR2 (FIG. 1A). Previous western analysis (Siegel et al., 2002) has shown this Ab binds with high affinity to a protein of >50 kDa (the anticipated size of the glycosylated protein (Uchida et al., 1999)), in both mouse and cat brains. Preincubation of the TR2-Ab with its immunizing peptide blocked binding.

To further confirm specificity of the anti-TR2 antibody, it was tested against recombinant TR2 proteins. Mammalian expression constructs were prepared for full length (TR2-FL) and the extracellular domain (TR2-ECD) of TR2, and expressed in CHO-S cells. As seen in FIG. 1B, cells transfected with the full length TR2 construct (lane 1) produce a strong signal at the anticipated size of TR2, similar to that of the endogenous mouse brain protein (lanes 5 and 6). No TR2 signal is observed in either vector control or untransfected cells (lanes 3 and 4, respectively). Similarly, cells transfected with the TR2-ECD construct show a signal at the predicted size of the extracellular domain. (The molecular weight of the transmembrane and intracellular domains is ˜6.7 kDa, therefore the extracellular domain should run slightly ahead of the full length TR2 protein.) The apparent lower expression level of TR2-ECD compared with full length TR2 may reflect the fact that the bulk of TR2-ECD was likely released into the culture medium (Uchida et al., 1999; Horie et al., 2000), and here only CHO-S cell pellet homogenates were examined.

The presence of a signal in the TR2 transfected cell lines, which is absent in the controls, as well as the shift in mobility between full length TR2 and its extracellular domain, demonstrate that the anti-TR2 antibody specifically recognizes TR2.

An isoform of TR2, TR1 exists, but it is unlikely that the TR2 antibody recognizes TR1. While the genes are ˜60% homologous at the DNA level, this drops to ˜50% identity at the protein level, and drops further to 40% (8 out of 20 amino acids) within the immunizing peptide. Further, the identical amino acids are not in a contiguous block, consequently reducing the likelihood of forming a common antigenic site for both TR2 and TR1. Mouse and human TR2 antigenic sites are identical.

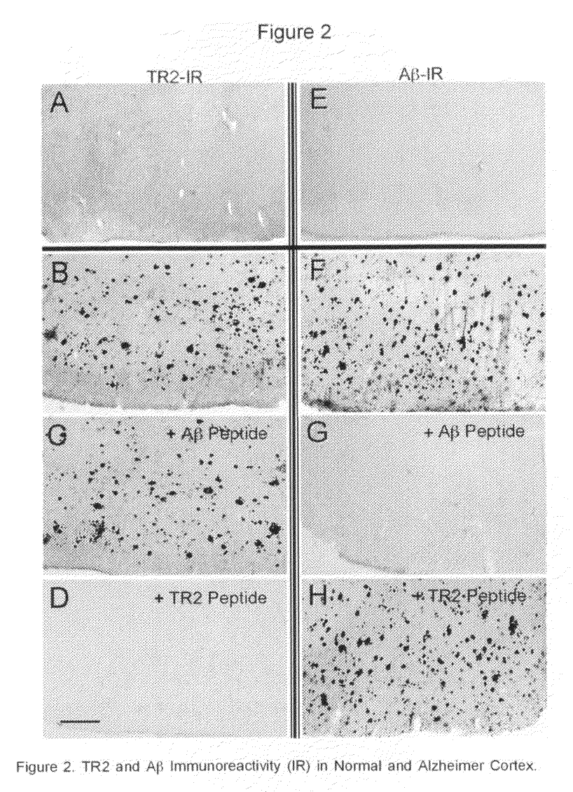

TR2 is Present in Alzheimer Disease Plaques: Immunohistochemistry (IHC) using the TR2-Ab revealed strong and abundant staining of amyloid plaques in AD brains (FIG. 2B). This staining was observed without pretreatment of the tissue with formic acid, which is typically used to enhance Aβ-Ab staining (Yamaguchi et al., (1988) Acta Neuropathol (Berl) 77, 113-119; Yamaguchi et al., (1988) Acta Neuropathol (Berl) 76, 541-549). To confirm that the TR2-Ab was indeed reacting with TR2 in plaques, and not cross-reacting with a similar epitope on the Aβ protein, a series of reciprocal antibody-peptide blocking experiments were performed in which each antibody was preincubated with its own or the other's immunizing peptide. For these experiments, all tissues were treated with formic acid, as Aβ immunoreactivity (IR) is poor in the absence of such treatment, while this treatment appeared to have only minimal affect on TR2-Ab binding (data not shown).

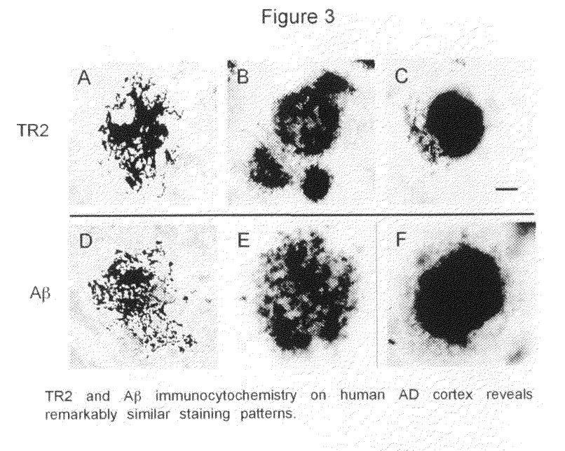

FIG. 2 demonstrates that while neither the TR2 nor Aβ antibodies display significant binding in normal aged human brain (FIGS. 2A and 2E respectively), both strongly bind to plaques in AD brain (FIGS. 2B and 2F respectively). These data also show that the number, distribution and appearance of plaques detected by TR2-Ab appear similar to those detected by the Aβ-Ab (see below and FIG. 3).

As expected, preincubation of each antibody with its immunizing peptide dramatically reduced antibody binding to plaques: TR2-Ab+TR2 peptide (FIG. 2D), Aβ-Ab+Aβ peptide (FIG. 2G). To demonstrate that the TR2 and Aβ antibodies are not recognizing a common epitope with similar three dimensional structure, each antibody was preincubated with the other's immunizing peptide. Neither TR2 nor Aβ antibody binding was blocked by this treatment: TR2-Ab+Aβ peptide (FIG. 2C), Aβ-Ab+TR-2 peptide (FIG. 2H).

TR2 and Aβ Plaque Morphologies are Similar: Classic plaque morphologies identified in AD brains using Aβ antibody are also observed with the TR2 antibody. FIG. 3 shows examples of AD plaques stained for both TR2 and Aβ. Diffuse amorphous deposits and mature plaques with and without dense amyloid core are observed. Morphologically TR2 and Aβ plaques appear indistinguishable.

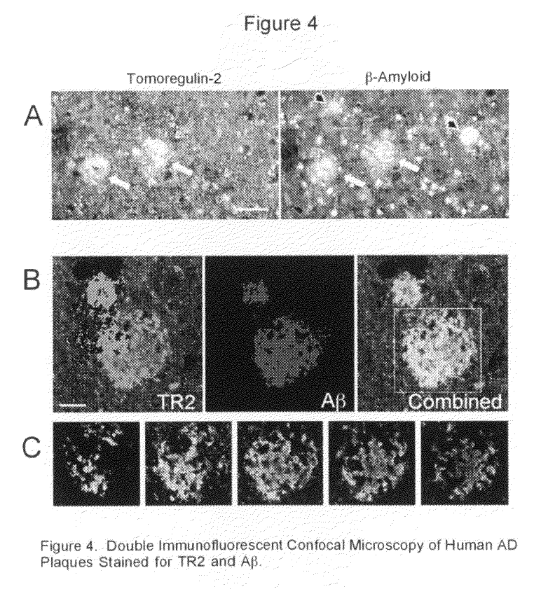

TR2 is Found in Many Aβ Positive Plaques and is Distributed Throughout Plaque Cores: Double immunofluorescence staining of AD tissue with TR2 and Aβ antibodies revealed that not all Aβ plaques are TR2 positive. An example is shown in FIG. 4A, in which two of four Aβ positive plaques are also positive for TR2.

In plaques positive for both TR2 and Aβ, TR2 is found distributed throughout the plaque. FIG. 4B shows two AD plaques stained with TR2 (green), and Aβ (red). The combined images reveal that TR2 and Aβ are distributed evenly throughout the plaque (yellow), although TR2 appears to surround the surface of the plaque as well.

The lower panel of this figure (4C) focuses on the larger of the two plaques, outlined within the white box in the combined image in 4B. It is a series of optical planes taken through the Z-axis of the plaque. Again, TR and Aβ immunostaining overlap (yellow) throughout the plaque, although distinct areas of green and red are evident. Again, as observed in FIG. 4B, TR2-IR appears to surround the plaque's surface in most planes.

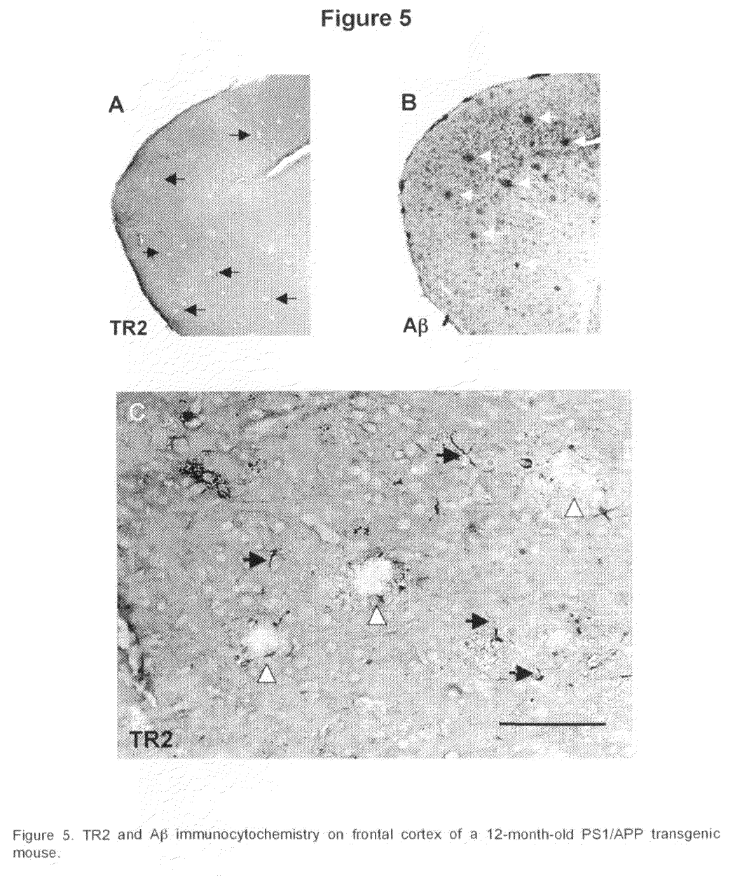

TR2 Does Not Stain Plaques in the PS I/APP Mouse Model of AD: In the PS1/APP mouse model of AD, Aβ immunoreactive plaques were readily identifiable throughout the brain, but no TR2 positive plaques were seen (FIG. 5). In fact, plaque-like areas were observed where TR2 staining seemed to be excluded (FIG. 5A). These TR2-negitive “ghost” plaques were often surrounded by TR2-positive process-like structures similar in appearance to dystrophic neurites (FIG. 5C). Also observed in the neuropil, were TR2 immunoreactive structures similar to neuropil threads. These process-like structures could be neuritic in origin, or associated with reactive astrocytes or microglia.

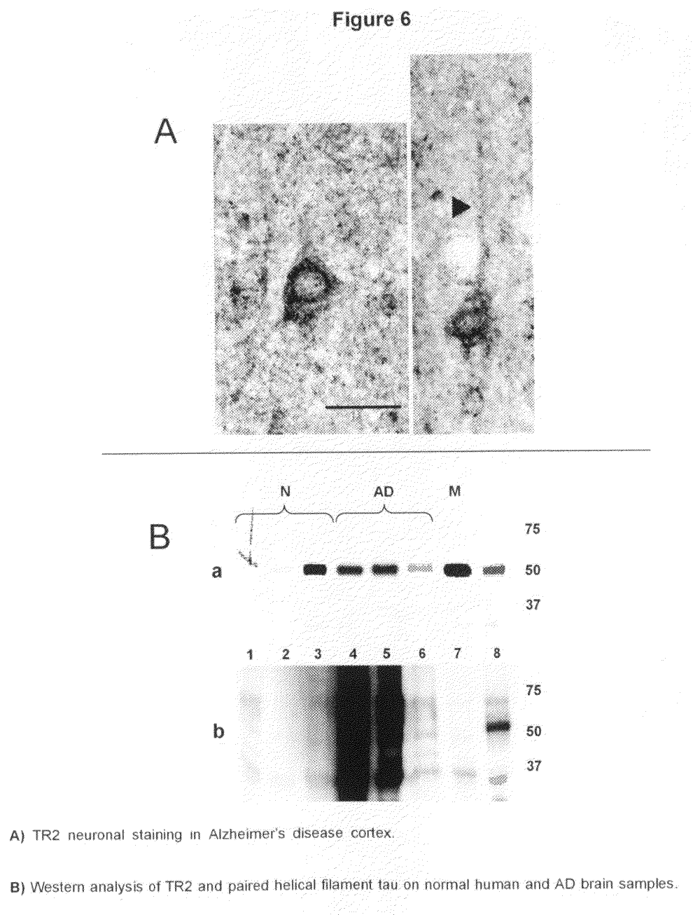

TR2 is Present in Neurons: FIG. 6A shows two pyramidal neurons from AD cortex with punctate staining in cell bodies and proximal processes, and for some distance along the apical dendrite in the neuron at right (arrowhead). Similar staining was observed in normal control brain (not shown).

The amount of TR2 in normal aged and AD brains was evaluated by western blot. FIG. 6B compares samples of three aged normal brains with three cases of severe AD, and one case of mild AD. To ascertain the extent of disease in these cases at the biochemical level, samples were also probed with antibodies to CP13, an antibody to paired helical filament which recognizes an AD specific form of tau, and correlates well with the later stages of AD (Davies, (1999) Characterization and use of monoclonal antibodies to tau and PHF-tau, in Methods in Molecular Medicine Alzheimer's Disease: Methods and Protocols (Hooper N. A., ed.), pp 361-374. Humana Press, Totowa, N.J.; Duff et al., (2000) Neurobiol Dis 7, 87-98).

In all cases 10 μg of protein were loaded per lane; duplicate gels were run. As may be seen in FIG. 6B-a, two of the severe cases of AD (lanes 4 & 5) and the mild case (lane 7) show strong TR2 signals. However, one AD case (lane 6) had significantly less TR2 than the other AD cases, and one normal aged brain (lane 3) revealed relatively high levels of TR2. The remaining two normal cases showed only faint TR2 signals.

FIG. 6B-b shows that the two AD cases with the greatest levels of TR2 also possess the greatest amounts of PHF (lanes 4 & 5), and that the AD case with the least amount of TR2 has the least amount of PHF (lane 6). However, the mild AD case (lane 7), which had a level of TR2 on par with the highest levels found in the severe AD cases (6B-a, lanes 4 & 5), had, as expected, less PHF than the severe cases (lanes 4 & 5). In addition, the aged normal case, which had significant amounts of TR2 (6B-a, lane 3), had PHF levels comparable to the other aged normal brains (lanes 1 & 2). Lanes 8 in both FIGS. 6B-a and 6B-b are adult mouse brain and were used as positive controls (Siegel et al., 2000).

### Discussion

The data presented demonstrate that TR2 is present both extensively within individual plaques and pervasively in plaques throughout Alzheimer's disease cortices. TR2 plaque morphologies appear identical to Aβ plaques and range from diffuse deposits to classic neuritic plaques with and without dense cores. A more detailed, quantitative study will be necessary to determine the percentage of amyloid plaques that are TR2 positive, and what, if any, differences exist between TR2/Aβ and Aβ-only plaques with respect to size, quality (number of diffuse vs. neuritic plaques), and brain distribution. Double label immunofluorescent confocal microscopy reveals that TR2 and Aβ are intimately associated throughout plaques, suggesting that TR2 is present at the onset of plaque formation and continues to accumulate with Aβ as plaques develop. In this regard TR2 appears to differ from many plaque-associated proteins that are often found on the periphery of plaques (Arai et al., (1990) Proc Natl Acad Sci USA 87, 2249-2253; Chaudhury et al., (2003) J Neuropathol Exp Neurol 62, 42-54; Culvenor et al., (1999) Am J Pathol 155, 1173-1181; Hashimoto et al., (2002) Embo J 21, 1524-1534; Schmidt et al., (1991) Lab Invest 64, 352-357). This, however, is not a universal finding as ApoE, for example, is also found throughout plaques (Nishiyama et al., (1997) Brain Res 750, 20-24).

The presence of non-Aβ proteins in some but not all Aβ plaques is a common observation, and the general absence of non-Aβ proteins from plaques in normal aged brains has led to the hypothesis that there is a qualitative difference between plaques found in normal aging brains (typically a non-fibrillar form of Aβ (Selkoe, (2001) Physiol Rev 81, 741-766)) and those found in patients with AD (fibrillar Aβ). Consequently, it has been proposed that non-Aβ proteins may play an active role in Aβ deposition in AD (Afagh et al., (1996) Expt'l Neurol 138, 22-32; Hashimoto et al., (2002) Embo J 21, 1524-1534).

TR2 is present in neurons in both AD and normal control brains, and appears as small puncta within cell bodies and proximal processes, and perhaps in the nucleus. This staining is similar to what is observed in normal adult mouse and cat neurons (Siegel et al., (2002) Int J Dev Neurosci 20,373-389), although cell morphology in human tissue is not as well defined and cell processes do not appear as long. These differences, however, likely result from the long delays in obtaining human tissues after death and their subsequent storage in formalin. For mouse and cat samples, animals (following deep sedation) are perfused with ice cold PBS followed by ice cold PFA; brains are kept overnight in PFA at 4° C., and finally transferred to ice cold PBS and stored at 4° C. (Siegel et al., 2002). The amount of TR2 perinuclear staining in AD, however, seems greater than what is observed in adult mouse and cat brains (Siegel et al., 2002). Confocal microscopy of cultured mouse primary cortical neurons demonstrates that TR2-immunoreactive puncta are inside the cell where they appear to be vesicles, perhaps translocating newly synthesized TR2 to the cell surface, and/or activated (cleaved TR2 extracellular domain) to the nucleus where it may function in transcriptional regulation (Siegel et al. 2002, manuscript in preparation). This trafficking may offer hints to the possible role of TR2 in the pathogenesis of AD (see below).

Like Aβ, the amount of TR2, as determined by western blot, does not correlate with clinical presentation in late stage AD (Tiraboschi et al., (2004) Neurology 62, 1984-1989). However, the appearance of elevated levels of TR2 in mild AD and apparently normal aged brain (FIG. 6B-a lanes 7 & 3 respectively) suggest that TR2 is an indicator of early disease processes.

TR2 positive plaques are found extensively in AD cortices. These plaques share the same morphologies as Aβ plaques and TR2 is present throughout them. The fundamental questions posed by these data are: What is the significance of TR2 in the overall pathogenesis of AD, and, through what mechanisms might TR2 contribute to plaque formation? The biochemical nature of TR2, its function, and normal subcellular location point to possible mechanisms. These mechanisms incorporate several processes already believed to contribute to AD, in particular the immune response and vesicle transport.

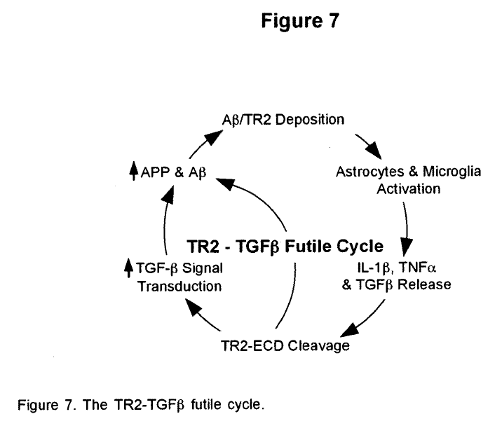

With respect to the immune response, deposition of even small amounts of Aβ have been shown to activate astrocytes and microglia, which, in turn, release proinflammatory cytokines interleukin 1β (IL-1β) and tumor necrosis factor α (TNF-α) as well as TGF-β (Akiyama et al., (2000) Neurobiol Aging 21, 383-421; Benveniste et al., (2001) Neurochem Int 39, 381-391; Cacquevel et al., (2004) Curr Drug Targets 5, 529-534; Mrak and Griffin, (2005) Neurobiol Aging 26, 349-354; Tarkowski, (2002) Curr Drug Targets Inflamm Allergy 1, 193-200; Wegiel et al., (2001) Neurobiol Aging 22, 49-61; Wyss-Coray, (2004) J Mol Neurosci 24, 149-153). TGF-β is believed to contribute to plaque formation because it stimulates APP expression (through activation of the Smad3 transcription factor pathway (Docagne et al., (2004) Biochem J 383, 393-399)) and, consequently, Aβ production (Amara et al., (1999) Brain Res Mol Brain Res 71, 42-49; Burton et al., (2002) Biochem Biophys Res Commun 295, 702-712; Harris-White et al., (1998) J Neurosci 18, 10366-10374; Lahiri et al., (2003) J Alzheimers Dis 5, 81-90). TR2 may augment this pathway by increasing TGF-β signaling. Tomoregulin-1 (TR1), the only other member of the tomoregulin family, inhibits TGF-β signaling by blocking TGF-β from binding to its receptor (Chang et al., (2003) Dev Biol 255, 1-11; Harms and Chang, (2003) Genes Dev 17, 2624-2629). However, when the TR1 extracellular domain is released from its transmembrane anchor, it allows TGF-β signaling to occur. TR2 is believed to function in a similar way. Of particular interest with respect to AD, however, is the fact that IL-1β and TNF-α stimulate the release of the TR2 extracellular domain (Lin et al., (2003) Life Sci 73, 1617-1627). This has two unfortunate consequences. First, it would allow increased TGF-β signaling and consequently increased Aβ production. Second, it would make available increased amounts of the TR2 extracellular domain for binding to and depositing with Aβ. This could initiate a “futile” cycle (FIG. 7) where increasing plaque load would stimulate more astrocytes and microglia. These, in turn, would release more IL-1β, TNF-α and TGF-β, causing the release of TR2-ECD, increased TGF-β signaling, increased Aβ production and more TR2/Aβ plaque formation continuing the cycle. Importantly, the 5′ UTR of TR2 appears to have a Smad3 response element similar to that of APP (Lahiri et al., (2003) J Alzheimers Dis 5, 81-90), which, if active, would only aggravate the futile cycle by simultaneously increasing expression of TR2 and APP, and consequently, plaque load. If the inflammatory response does contributes to TR2/Aβ plaque formation, the absence of TR2 plaques in the PS1/APP mouse model might, in part, be attributed to the more limited immune response observed in mice (Selkoe, (2001) Physiol Rev 81, 741-766).

It is also believed that TR2 interacts with Aβ prior to reaching the plasmamembrane. APP has been shown to travel in both anterograde and retrograde directions in vesicles within neurites (Stokin et al., (2005) Science 307, 1282-1288; Yamazaki et al., (1995) J Cell Biol 129, 431-442). APP processing to Aβ occurs within these vesicles (Amaratunga and Fine, (1995) J Biol Chem 270, 17268-17272) where it is believed to contribute to dystrophic neurite and plaque formation early in the disease process (Stokin et al., 2005). TR2 is observed as numerous small puncta within neurites that appear vesicular in nature and may represent anterograde movement of newly synthesized TR2 to the cell surface and/or retrograde translocation of activated TR2 to the nucleus where it may function in dendrite initiation (Siegel et al., (2002) Int J Dev Neurosci 20, 373-389), or as a trophic factor (Horie et al., (2000) Genomics 67, 146-152). If TR2 and APP are present in the same vesicles, TR2/Aβ complexes could form within them, contributing to dystrophic neurite formation by disrupting vesicle trafficking (Stokin et al., 2005), and to plaque formation if these vesicles release their contents into the neuropil. It is worth noting that TR2 may function in both normal and ectopic dendrite initiation (Horie et al., 2000, Siegel et al., 2002), and could contribute to dystrophic neurite initiation in AD through a similar mechanism.

TR2 is located on chromosome 2q32.3. The only AD candidate gene in this region, divalent cation transporter SLC11A1, is located at 2q35 and has recently been shown by linkage analysis not to be associated with AD (Jamieson, et al., (2005) Neurosci Lett 374, 124-128). Interestingly, linkage analysis does suggest a strong genetic influence of chromosome 2q36-37 on Parkinson's disease (Pankratz et al., (2003) Am J Hum Genet 72, 1053-1057; Pankratz, et al., (2003) Hum Mol Genet 12, 2599-2608). Evidence that TR2 may be a trophic factor and dendritogenic agent for mesencephalic dopaminergic neurons (Horie et al., (2000) Genomics 67, 146-152) makes TR2 an attractive candidate gene affecting Parkinson's as well as Alzheimer's disease.

The extensive presence of TR2 in AD plaques is a new finding. The structure, function and subcellular location of TR2 suggest that its presence in plaques is not simply a non-specific co-deposition of protein, but part of the pathogenic process. It is not clear at present whether TR2 and Aβ (or APP) participate in a normal functional complex, or whether these two proteins simply have an unfortunate affinity for each other that only becomes manifest in the unusual circumstances of AD. However, it is clear that a new and major component of amyloid plaques has been identified, and its presence in these structures offers new opportunities to develop new therapeutic approaches toward preventing it, all of which are included in the presently described invention.

### Example 2

Our data, demonstrating that TR2 is found extensively in Alzheimer plaques and appears closely associated with Aβ in plaque cores, suggests that TR2 may play a previously unknown, but fundamental, role in amyloid deposition.

Immunocytochemistry of Alzheimer brain with TR2 antibody (TR2-Ab) reveals extensive labeling of senile plaques (FIG. 5); confocal microscopy demonstrates that TR2 and Aβ are closely associated within plaque cores (FIG. 7). As our antibody recognizes an epitope on the amino-most follistatin module of the TR2 extracellular domain (TR2-ECD) (FIG. 1A), at least this portion of TR2 is present in plaques. Because the entire extracellular domain of TR2 can normally be cleaved by cell surface proteases (as are numerous other cell surface proteins such as APP, Notch, CD44, erbB4 and the erbB4 receptor, it seems likely that at least the TR2-ECD is present in plaques. Western blots of AD brain samples probed with our TR2-Ab appear to bear this out. We see a band, migrating at the predicted size of the TR2-ECD only in AD samples. Thus, if TR2 binds Aβ, it seems that the Aβ binding site is in the TR2-ECD.

### Example2a

### TR2-ECD Binding Aβ—Immunoprecipitation Experiments

The simplest way to determine if TR2-ECD binds Aβ is to mix the two proteins in solution and perform immunoprecipitation (IP) experiments. In these experiments we will use conditioned medium from our TR2-ECD stably transformed CHO cell line (FIG. 1B) as a source of TR2-ECD. The TR2-ECD construct was prepared from our full-length cDNA and includes the entire extracellular domain (signal peptide, two follistatin motifs and the EGF module). Cells will be grown in defined medium to limit the number of extraneous proteins found in serum. Conditioned medium will be separated from cells by centrifugation, and levels of TR2-ECD determined by western blot. The Aβ-42 peptide will be purchased from Bachem Bioscience, King of Prussia, Pa. Reciprocal IP experiments will be done using our TR2-Ab and anti-Aβ-42 antibody (6E10) from Signet Labs, Dedham, Mass. Preliminary experiments will use equimolar amounts of TR2-ECD and Aβ342 at concentrations of 5 μM under our standard IP conditions. Standard controls will be done, e.g. IPs with and without primary antibodies and primary antigens, and unrelated control protein IP with CTLA4-Fc (cytotoxic T lymphocyte antigen-4 with a human IgG fc-tag).

Co-precipitation of Aβ with TR2-ECD and/or TR2-ECD with Aβ would argue that TR2-ECD and Aβ do interact and are co-deposit in amyloid plaques. This result would support our hypothesis that TR2 plays a fundamental role amyloid plaque formation. It is possible, however, that either one, or both, IPs could fail, if the precipitating antibody were blocked from binding its epitope because that site was occupied by the other protein. For example, if Aβ were bound to TR2-ECD in such a way that the Aβ antibody could no longer bind its recognition site, immunoprecipitation of Aβ with its antibody could not occur. This eventuality can be controlled for by using epitope tagged Aβ or TR2-ECD proteins in the immunoprecipitation experiments. For example, we have fusion constructs for both Aβ (courtesy of Dr. L. D'Adamio, collaborator) and TR2-ECD tagged with the human IgG fc epitope. Thus, if immunoprecipitation experiments with the TR2-ECD or Aβ antibodies fail, we will prepare fc-tagged TR2-ECD or fc-tagged Aβ proteins, and use these for immunoprecipitation. We chose not to do initial IP experiments with tagged proteins because the tags themselves can interfere with protein binding. For example, an fc tag on TR2-ECD could prevent it from binding Aβ and vice versa. It should be noted, that we have two constructs for each protein, one tagged at the amino terminus, and the second tagged at the carboxy terminus. Thus, if we did not detect binding of amino fc tagged TR2-ECD to Aβ, we would try carboxy tagged TR2-ECD. These reagents should allow us to come to a definitive conclusion on whether TR2-ECD and Aβ bind each other.

### Example 2b

### Determination of which Parts of the TR2-ECD Bind Aβ

The goal of this experiment is to precisely map that portion of the TR2 extracellular domain that is responsible for binding Aβ. This will be done using deletion constructs of the TR2-ECD from which different modules have been removed, and testing the ability of these constructs to bind Aβ in immunoprecipitation assays. The results of Experiment 1 above will define many of the assay parameters employed here, including the concentrations of TR2-ECD and Aβ, as well as whether or not we can use our TR2-ECD antibody in initial experiments, or whether we need to use fc tagged fusion proteins. Thus, for example, assuming that our TR2 antibody can pull down Aβ as described in Experiment 1, we will then begin dissecting the Aβ binding domain of TR2-ECD through serial deletion of modules in the TR2-ECD beginning at the carboxy terminal. The first construct tested will lack the EGF domain. If this construct were able to IP Aβ, we would then remove the carboxy most follistatin-like domain and test the ability of the remaining portion of TR2-ECD to precipitate Aβ. These construct are already prepared. If however, we lose Aβ binding ability with the removal of one of these modules, we will then prepare a construct with that module tagged with fc to test whether it posses the Aβ binding site. Again, we will, if necessary, prepare these constructs with the fc tag at the carboxy and amino termini. These large truncation experiments should help us identify the Aβ binding region.

It may be, however, that TR2-ECD binding of Aβ is not a normal physiological property of TR2, but rather an unfortunate consequence of the two proteins being released in the same place and time as the result of a complex pathological pathway that allows them to come together and form plaques. Thus, there is no reason to assume that Aβ binds to a particular module (EGF or follistatin) of TR2 (unlike TR2 and TGF-β). Consequently, once we have established the portion of the TR2-ECD that possesses the Aβ binding site, we will further refine the binding area with additional deletion constructs of the identified binding domain. Our final goal is to be able to define the region with enough specificity so as to be able to make specific point mutations and determine their effects on Aβ binding.

It is anticipated that from these experiments the precise Aβ binding domain in the TR2-ECD will be identified. Careful analysis of that region may show that deletions, or perhaps even specific substitutions of a few amino acids will be able to abolish or greatly diminish TR2 binding affinity for Aβ. This knowledge could be helpful for understanding the pathogenesis of AD. For example, normally occurring amino acid substitutions in the Aβ binding domain of TR2 might offer some protection against plaque formation, and be a positive genetic marker against AD. Other substitutions could increase the binding affinity of TR2 for Aβ and adversely affect the disease process by enhancing plaque formation. Genetic analysis of the Aβ binding domain of TR2 in AD patients could prove extremely interesting, as it could identify natural polymorphisms that predispose patients for AD. This information could be helpful for identifying people at risk for the disease.

Knowledge of the Aβ binding domain could have potential therapeutic effects as well. The vast majority of plaques we have observed in the eight patients examined to date were TR2 positive. Thus, if TR2 plays a role in Aβ deposition, inhibiting the interaction between Aβ and TR2 could reduce Aβ/TR2 plaque formation. Knowing the biochemical attributes of the TR2 Aβ binding domain would permit the establishment of a high throughput drug discovery assay system to test natural substances, as well as allow for rational, directed synthesis of interfering therapeutic agents.

Finally, we are aware that the TR2 binding domain for Aβ could result from the folding of the TR2 extracellular domain, bringing together distant parts of the molecule to form the binding site. Our planned deletion analysis would detect this possibility. If, for example, deletion of the carboxy terminus eliminated or diminished Aβ binding, and deletion of the amino terminus also eliminated or diminished binding, it would suggest that these two portions of the protein come together to form the Aβ binding site. Further analysis, such as more refined deletions or substations in those areas, or removal and/or substitution of intervening peptides, would aid in defining the binding site. While these possibilities might make identifying the Aβ binding domain more challenging, they would not prevent us from fully defining it. Nor would this outcome diminish the value of identifying polymorphisms in AD patients that might act markers for predisposition to the disease, nor would it diminish the ability to find and exploit therapeutic agents capable blocking TR2 Aβ binding.

### Example 2 C

### Determining Whether the TR2-ECD and Aβ can Form Aggregates

The experiments above are designed to determine if TR2-ECD and Aβ can bind to each other, and, if so, to define the TR2 Aβ binding domain. This experiment is designed to determine if TR2-ECD and Aβ naturally form aggregates in solution, and if so, does TR2 increase the rate and/or the amount of Aβ aggregation.

Monomers of Aβ can aggregate in solution to form insoluble precipitates. Assay systems have been established to monitor Aβ aggregation and have been used to screen for substances that can contribute to, or inhibit plaques formation. Here we propose to use the Congo red assay system in conjunction with denaturing and non-denaturing polyacrylamide gel electrophoresis (PAGE) to evaluate the ability of TR2-ECD to enhance Aβ aggregation. These methods have several advantages. The Congo red system measures Aβ aggregation by following changes in dye absorption in a relatively simple assay and is an accurate reflection of aggregation. (We will use the equation described by Klunk et al. Anal. Biochem. 266:66-76 (1999) to measure dye absorption.) The use of denaturing and non-denaturing PAGE allows us to distinguish between weakly associated protein interactions, and insoluble aggregates that may be more reflective of the compact plaques found in AD.

We will follow the method of Klug et al. Eur. J Biochem. 270:4282-4293 (2003). Stock solutions of Aβ will be dissolved in dimethylsulfoxide, kept at −80° C., and diluted into assay buffer immediately prior to use in the neutral buffer system. This will limit Aβ self aggregation prior to use. We will purify TR2-ECD from our HIS labeled constructs using Ni chelation chromatography, and check quality by PAGE, western and silver staining (for contaminating proteins) (Note: We have already prepared two TR2-ECD constructs, one with a HIS tag at the amino terminal, the second at carboxy terminal in the event that one tag interferes with Aβ binding.) Initial experiments, including time course, Aβ/Congo red ratio, etc., will replicate the condition used by Klug et al. to ensure that the assay system is working properly. Controls will include TR2-ECD separately to check for TR2-ECD self aggregation. Once the assay is established, we will perform TR2-ECD concentration curves to measure changes in both the rate of Aβ aggregation and the amount precipitated. Aβ-only aggregations will be run in each experiment to establish baseline levels for comparison with TR2-Aβ aggregation. TR2 and Aβ will be detected by western blot. Experiments will be done in triplicate and the amounts of protein precipitated quantified by NIH Image analysis software.

These experiments will help define the role of TR2 in amyloid plaque formation. It is anticipated that TR2-ECD will bind to Aβ and precipitate with it. This would demonstrate the two proteins can form plaques, and suggest that if TR2 were not present, that plaque load would be less, and, possibly, disease progression slowed. Additionally, we will be able to determine if TR2-ECD i) accelerates Aβ aggregation, i.e., functions as a nucleating agent or catalyst, and ii) increases the amount of Aβ precipitated. These latter two points are important, as they would suggest that the presence of TR2 exacerbates the disease process. Indeed, it may be that the difference between the Aβ and TR2/Aβ plaques we observe in AD brain define two different disease processes, or the disease at different times in its progression. Alternatively, plaques that contain only Aβ may represent an early stage of plaque formation, which is subsequently followed by incorporation of TR2 and consequently increased plaque load. This would argue in favor of our futile cycle hypothesis.

Finally, we will use the information obtained from Example 2b above to see if the entire TR2-ECD is necessary for Aβ/TR2 aggregation, or whether smaller parts of the molecule are sufficient. It may be that while only specific sections of TR2 are capable of binding Aβ, the entire molecule is necessary to form the precipitating aggregate, either because there are multiple Aβ binding sites on TR2, or because TR2-TR2 interactions are also important to plaque formation. These experiments will repeat the TR2-ECD concentration curve described above with specific TR2-ECD peptides.

### Example 3

### Example 3a

### Determining if Full-Length Tomoregulin-2 can Block TGF-β/Smad3 Signal Transduction

TFG-β signal transduction is initiated when TGF-β binds its receptor and activates the Smad transcription factors (FIG. 8). These then translocate to the nucleus where they bind TGF-β responsive elements and regulate gene transcription. TGF-β stimulation of APP expression in astrocytes has been shown to function through activation of Smad3. This experiment will test whether tomoregulin-2 can block TGF-β1 activation of Smad3. We will use a CHO cell assay system, similar the one employed by Lesné et al. using astrocytes (See Burton, T et al. Biochem Biophys Res Commun 295: 702-712 (2002), to measure TGF-β receptor activation. (We are aware that there are different TGF-β isoforms and that the strength of their down stream signals, and ability to interact with modifying proteins such as TR2 vary depending on the receptor subtypes expressed. However, CHO cells are known to be responsive to TGF-β (see below), but we are prepared to examine other TGF-β isotypes depending on results.)

CHO cells express TGF-β type I and II receptor subunits, and respond to TGF-β by activating the Smad3 transcription factor. CHO cells do not express tomoregulin-2 (FIG. 1B). These attributes make CHO cells ideal for testing the ability of TR-2 to inhibit TGF-β signaling. In this in vivo assay system, we will measure the level of TGF-β stimulated signal transduction in the presence and absence of TR2 through a Smad3 activated reporter gene, firefly luciferase. This system has four distinct advantages: i) it will allow for a straightforward determination of the effects of TR2 on TGF-β initiated signal transduction, ii) the luciferase assay is sensitive, quantitative and well controlled, and will allow for accurate measurements, iii) if TR2 is shown to block TGF-β signal transduction, this system can be used to determine which structural motifs of the TR2 extracellular domain are involved, iv) this system can also be used to determine whether the TR2 extracellular domain alone is capable of blocking TGF-β signal transduction.

TGF-β REPORTER SYSTEM: Transcription factor reporter assays, such as the Smad3 reporter system to be used here, are designed to detect cellular responses to biological stimuli, such as receptor activation following ligand binding. Typically, a transcription factor DNA binding domain (response element) is linked upstream to a reporter gene and the construct transfected into cells. On receptor stimulation, a transcription factor is activated which binds to the response element on the reporter construct and initiates synthesis of the reporter gene product. An important aspect of any reporter system is an internal control. This is typically a second reporter, running off a low expressing, constitutive response element that can be measured independently of the primary (experimental) reporter. The control reporter standardizes samples within an experiment so that they may be legitimately compared to one another. This is achieved by a simple ratio of experimental/control reporter values within each sample, and corrects for sample variations due to transfection efficiencies and cell numbers.

Our TGF-β reporter system will use the 21 base pair Smad3 response element identified in the APP gene linked to the bioluminescent reporter firefly luciferase (pGL4, Promega). TGF-β stimulation of its receptor will lead to activation of the Smad3 transcription factor, which in turn, will bind to the Smad3 response element and initiate synthesis of the luciferase gene product. Luciferase activity is detected by conversion of a chemoluminescent substrate. Control reporter will be sea pansy (Renilla reniformis) luciferase running off the relatively weak, constitutive HSV-thymidine kinase promoter (phRL-TK(Int-), Promega). Sea pansy luciferase catalyzes a different substrate than firefly luciferase, and, consequently, the two reporters do not interfere with one another. The two activities are checked sequentially, after addition of the second substrate.

Luciferases are extremely sensitive reporters and can be detected in the fM range. Sequences of both the firefly and sea pansy vectors have been modified from wild type to i) reduce background expression by eliminating anomalous transcription factor recognition sites in the vector, and ii) increase sensitivity by optimizing codon usage for mammalian cells.

To determine if TR2 can block TGF-β initiated signal transduction, we will measure Smad3 reporter activity in CHO cells in the presence and absence of TR2. In these experiments the CHO parental and CHO-TR2 expressing cell lines will be transfected with the Smad3-firefly luciferase reporter (Smad3-FFLuc) and control sea pansy luciferase (C-SPLuc) constructs. After recovery, cells will be stimulated with TGF-β and reporter and control expression measured. Preliminary experiments will establish all assay parameters including: amount of constructs to be transfected, recovery time necessary to reach maximal reporter expression, concentration curve for TGF-β.

It is anticipated that the parental CHO cell line will show a positive dose response to increasing levels of TGF-β, and that the CHO-TR2 cell line will show a limited response. If predicted results are obtained, they will support our hypothesis that TR2, like TR1, functions by blocking TGF-β signal transduction.

Interpretation of results requires a thorough understanding of the assay system and methods to evaluate and control for potentially confounding variables. Three such variables will be evaluated: i) endogenous levels of TGF-β ligand, ii) endogenous proteases that may cleave the TR2-ECD, and iii) the binding affinity of the TR2-ECD for TGF-β.

i) Endogenous TGF-β: While CHO cells express functional TGF-β receptor, they express little TGF-β ligand. Consequently, they have a low baseline level of activity and are used to study TGF-β signal transduction by exogenously added ligand. Baseline levels of reporter activity (i.e. measured the absence of exogenously added TGF-β) will be part of the standard assay. If necessary, endogenous TGF-β can be reduced by rinsing cells with fresh media prior to assay.

ii) Endogenous Proteases: It is possible that endogenous proteases could cleave the extracellular domain of TR2, preventing it from blocking the actions of TGF-β. This, however, seems unlikely as we find large quantities of full length TR2 in the membrane fraction of homogenates of our CHO-TR2 cell line (FIG. 1B). Although unlikely, it is possible that exposure of these cells to TGF-β could induce expression of proteases that might cleave the TR2-ECD from the full-length protein. Consequently, we will measure, by western blot, the levels of TR2 and TR2-ECD in the membrane and supernatant fractions of cells treated with TGF-β to confirm that ECD is not being cleaved. If a significant amount of the TR2-ECD is found in the supernatant, several precautions can be taken to remove it prior to assay, as well as limit cleavage during assay. First, cells can be washed with culture medium to remove any cleaved TR2-ECD immediately before TGF-β treatment. Second, protease inhibitors, including those shown to block cytokine induced metalloproteases from acting on TR2 can be added to the medium. Again, from our experience, endogenous cleavage of TR2-ECD in CHO cell does not seem likely, and can be tested and controlled for.

iii) TR2-ECD/TGF-β Binding: Although it has been demonstrated that the TR1 extracellular domain can only block nodal and BMP2 (both TGF-β family members) signaling when attached to the cell membrane, it is possible that the free TR2-ECD may be able to inhibit TGF-β signaling. As designed, this assay system cannot distinguish between the effects of bound or free TR2 activity, but only whether TR2 can block TGF-β activated signal transduction. To determine whether the TR2-ECD can block TGF-β signaling, a separate assay will be used, as described in Experiment 3 below.

### Example 3b

### Determining which Parts of TR2 are Necessary for Inhibitory Activity

The inhibitory activity of TR1 against nodal signaling requires the entire extracellular domain (follistatin & EGF modules) to be present, and the molecule be anchored to the plasmalemma. The cytosolic domain is not required. To inhibit BMP2 activity, either the follistatin modules or the EGF module must be present, the molecule must be tether to the membrane, and the cytosolic domain is required. The experiments described below are designed to determine which parts of TR2 are necessary to block TGF-β signaling.

We have already prepared the necessary constructs for this work in the same mammalian expression vector used for full length TR2 (pCMV-Script, Stratagene). TR2 possesses, from the extracellular amino terminus to the cytosolic carboxy terminus, the following modules (FIG. 1A): signal peptide, two follistatin domains, the EGF domain, transmembrane domain, and the cytosolic domain. The five deletion constructs all possess the signal peptide and transmembrane domain, but lack one of the following motifs: 1) the first (amino-most) follistatin module, 2) the second follistatin module, 3) both follistatin modules, 4) the EGF module, and 5) the cytosolic domain. CHO cells will be transfected (separately) with each construct and we will test their response to TGF-β in a manner similar to what was done with the full-length construct in Example 3a above.

While it is not possible to know a priori which modules of TR2 are necessary to block TGF-β signal transduction, it is anticipated that, if TR2 is capable of blocking TGF-β signaling, then these experiments will be able to determine which modules are involved.

### Example 3c

### Determining if the TR2-ECD can Block TGF-β Signal Transduction

The assay system employed here will be similar to that used in Example 3a above, except that we will use only the CHO parental cell line. Cells will be stimulated with TGF-β in the presence and absence of purified TR2-ECD. We have made two TR2-ECD constructs for preparing TR2-ECD protein. They were made in the same mammalian expression vector used for full length TR2 (pCMV-Script, Stratagene). Each possess the signal peptide, two follistatin modules, the EGF motif, and a HIS tag for purification. One construct has the HIS tag at the carboxy terminus, the second at the amino terminus as it is not possible to know whether or not a HIS tag will interfere with function. (Note: If necessary, we can also use conditioned media from our TR2-ECD construct prepared without any tag (FIG. 1B). However, without a tag the protein cannot be easily purified.) The ability of TR2-ECD to block TGF-β signal transduction will again be measured by Smad3-FFLuc reporter activation; sea pansy luciferase will be used as control. Optimal TGF-β concentra-tion will have been determined in Example 3a. Potential inhibitory activity of TR2-ECD will be examined over a wide range of concentrations. TR2-ECD inhibitory activity will be tested in two ways: 1) TR2-ECD will be preincubated with cells to determine if it first needs to interact with the TGF-β receptor to block TGF-β ligand, and 2) TR2-ECD will be preincubated with TGF-β ligand to determine if a TR2-ECD/FGF-β complex is formed.

If TR2 functions in a manner similar to TR1, we would anticipate that the TR2-ECD will not block TGF-β signal transduction since TR2-ECD is not bound to the plasmalemma. Such a result would support our hypothesis that the TR2-ECD, once released, can no longer prevent TGF-β receptor activation and the subsequent amyloidogenic consequences of that activation. If, however, we observe lower levels of Smad3 activated FFLuc in TR2-ECD treated cells, it would imply that TR2-ECD is capable of blocking TGF-β signal transduction, either by directly binding to TGF-β ligand, or interfering with the TGF-β receptor. This interpretation would, of course, depend on the concentration at which TR2-ECD inhibition is observed. If it occurs at relatively high concentrations, it would suggest that the effect is either non-specific, or that the affinity of free TR2-ECD for TGF-β or its receptor, is far less than full length, membrane bound TR2.

### Methods

Constructs: Full length human TR2 cDNA was isolated from a human brain cDNA library (Invitrogen) using our mouse TR2 cDNA. The full length and extracellular domain of TR2 cDNAs were cloned into the mammalian expression vector pCMV-Script (Stratagene) by PCR of the original pBlueScript TR2 clone followed by ligation into the pCMV vector. This vector uses a CMV promoter for gene expression and has an SV40 polyadenylation signal downstream to ensure proper 3′ mRNA processing. Primers for amplification of full length TR2 were prepared with a Not I restriction site followed by a Kozak consensus sequence upstream of the 5′ start site, and a stop codon and EcoR I restriction site downstream of the 3′ end of TR2. For cloning the TR2 extracellular domain and its parts, the same 5′ primer was used, but in conjunction with a 3′ primer containing sequence adjacent to the extracellular side of the transmembrane domain, followed by a stop codon and EcoR I restriction site. Following PCR, amplicons were purified by Qiagen spin column chromatography, digested with Not I and EcoR I, and electrophoresed through an agarose gel. Bands of anticipated size were cut from the gel, purified with a Qiagen gel extraction kit, and cloned into the multiple cloning site of the pCMV vector. After transformation into E. coli (XL-1 Blues, Stratagene), plasmids were purified, sequences checked twice by sequencing.

For the Luciferase Reporter Assay System: We will use the method of Docagne et al. The APP TGF-β response element

(CCCGGGAGACGGCGGCGGTGG) (SEQ ID NO. 5) will be synthesized with Xho I and Hind III restriction site at the 5′ and 3′ end and ligated into the multiple cloning site on the firefly luciferase vector pGL4 (Promega). We will use the sea pansy (Renilla) luciferase vector pGL4.74 (hRluc/TK) as a reporter control. It possesses the herpes simplex virus thymidine kinase (HSV-TK) promoter upstream of sea pansy luciferase and provides low levels of constitutive expression in co-transfected cells. The two luciferases react with different substrates and are measured independently using Dual Luciferase Reporter Assay System (Promega) (see Specific Aim 2). One day following transfection cells are treated with TGF-β (1 ng/ml) and luciferase activity measured after 24 hrs. following cell lysis.

Cell Culture, Transfection, and Expression Of Recombinant Proteins: CHO-S cells (Gibco) are maintained in CD CHO medium (Gibco) supplemented with 8 mM L-glutamine, 0.1 mM sodium hypoxanthine, 0.016 mM thymidine for suspension cells, and Dulbecco's modified Eagle medium supplemented with 0.1 mM MEM non-essential amino acids and 10% fetal bovine serum for adherent cells. Cells are transfected with individual plasmids and empty vector control using Lipofectamine 2000 following manufacturer's instructions.

Purification of Recombinant Proteins From Conditioned Medium: Cells are spun at spun at 3,500 g×6 min. and supernatants collected. Before purification supernatant is spun again at 20,000×g×10 min. at 4° C. to remove debris, protein content is determined by Bradford (4). For fc tagged proteins, purification is done with Protein A covalently bound to agarose beads (Pierce). Supernatant is brought to neutral pH with 2× binding buffer (0.2 M sodium phosphate pH 7.0, 0.30 M sodium chloride, 10.0 mM sodium EDTA, 0.02% sodium azide, and protease inhibitors aprotinin 10 ug/ml, leupeptin 10 ug/ml, PMSF 1 mM) and passed through Protein A spin column. Note: preliminary experiments will be done to ensure no more than 60% of the column's binding capacity is loaded. Column is washed with 5 bed volumes of 1× loading buffer, and eluted in alkaline pH elution buffer (0.5 M ammonium acetate pH 3.0, and 0.01% sodium azide). Eluate is immediately neutralized with 0.5 M Tris base, and 0.01% sodium azide, and dialyzed in appropriate buffer: e.g. for IP, binding assays, etc. (For large scale production, we use column chromatography with peristaltic pump; all work is done at 4° C.). Purified proteins are quantified by Bradford (4) and checked for purity by silver stain and/western blot as previously described (4). For HIS tagged proteins Ni immobilized resin is used. Supernatants are treated the same as above. Samples are loaded onto spin columns in loading buffer (50 mM sodium phosphate pH 7.0, 300 mM NaCl) and wash with five bed volumes of the same buffer. Samples are eluted with imidazole elution buffer (50 mM sodium phosphate pH 7.0, 300 mM NaCl, 150 mM imidazole), and again dialyzed into appropriate buffer. Samples are quantified and checked for purity as described above.

Immunoprecipitation: Initial experiments will use conditioned medium from our CHO TR2-ECD cell line as a source for TR2-ECD (FIG. 1B). Medium will be spun at 20,000×g×10 min at 4° C. Protein will be quantified by Bradford, TR2-ECD concentrations estimated by silver stained PAGE gels scanned by NIH Image and check for purity by western. Fc tagged proteins will be prepared as described above (Purification of Recombinant Proteins From Conditioned Medium). The Aβ-42 peptide will be purchased from Bachem Bioscience, King of Prussia, Pa. We will use our affinity purified rabbit anti-TR2 Ab, anti-Aβ42 Ab (6E10) from Signet Labs, Dedham, Mass., and Pierce Protein A beads. EP binding buffer is 140 mM NaCl, 8 mM sodium phosphate, 2 mM potassium phosphate and 10 M KCl, 10 mM Tris, pH 7.4. In preliminary experiments, 5 μM of TR2-ECD and Aβ42 are combined and mixed with 2.5 μg of either our anti-TR2 Ab or anti-Aβ42 Ab and brought to 1 ml with appropriate volumes of H2O and 2×IP buffer and incubated for 1 hr. at 4° C. 50 μl of a well-washed 20% solution of Protein A beads in IP buffer is added and the reaction incubated overnight at 4° C. with gentle shaking. Samples are centrifuged at 16,000×g for 1 min., washed 3× with IP buffer, centrifuged again, and the supernatant removed. The Protein A pellet is resuspended in 30 μl SDS PAGE loading dye and heated to 100° C. for 5 min; 10 μl of each sample are loaded on a 10-20% gradient SDS PAGE gel. Following electrophoresis proteins are transferred to a PVDF membrane and visualized by western. Several important controls are performed. First, solutions of TR2-ECD and Aβ are incubated separately with their opposite Ab (i.e. TR2-ECD with anti-Aβ42 Ab and vise versa) to ensure that neither protein is binding non-specifically to the opposite Ab. Similarly, both proteins will be incubated separately with Protein A beads to demonstrate they are not binding to Protein A.

In the preceding specification, the invention has been described with reference to specific exemplary embodiments and examples thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense.

1. A method for the treatment of Alzheimer's disease comprising administering a composition to a human patient in need thereof, said composition comprising a protein having an amino acid sequence that corresponds to a portion of the extracellular domain of TR-2 and inhibits cleavage of a portion of the extracellular domain of tomoregulin-2 (TR-2) by binding to a TR-2 protease, thereby preventing the protease from binding and cleaving the extracellular domain of TR-2.

2. The method of claim 1, wherein the protein comprises an amino acid sequence corresponding to SEQ ID No. 1.

3. The method of claim 1, wherein the protein comprises a partial amino acid sequence corresponding to SEQ ID No. 1.

4. The method of claim 1, wherein the protein comprises a modified amino acid sequence corresponding to SEQ ID No. 1.

5-8. (canceled)

9. A method for inhibiting formation of plaques associated with Alzheimer's disease that comprise beta-amyloid and a portion of TR-2 in a human patient comprising administering to the human patient an effective amount an antibody specific for a portion of the extracellular domain of TR-2, wherein the antibody binds to a portion of the extracellular domain of TR-2, thereby preventing beta amyloid from binding to a portion of the extracellular domain of TR-2 to form plaques.

10. The method of claim 9, wherein the antibody binds to a portion of the extracellular domain of TR-2, thereby initiating an immune response and removal of the plaques.

11. A method for diagnosing and/or monitoring Alzheimer's disease in a human patient comprising:
a) taking a sample of tissue or fluid from the human patient;
b) adding an antibody specific for a portion of TR-2 to the sample of tissue or fluid to created a mixture;
c) testing for binding of the antibody to a portion of TR-2;
detection of bound antibody being indicative of Alzheimer's disease.

12. The method of claim 11, wherein the antibody is specific for a portion of the extracellular domain of TR-2.

13. The method of claim 12, wherein the antibody is specific for a portion of the extracellular domain of the TR-2 that has an amino acid sequence corresponding to SEQ ID No. 1.

14. The method of claim 11 for monitoring Alzheimer's disease progression further comprising:
a) quantifying the amount of TR-2 present in said first sample;
b) taking a second sample of tissue or fluid from the human patient at a point in time after the taking of the first sample;
c) adding the antibody specific for a portion of TR-2 to said second sample;
d) testing for binding of the antibody to TR-2 and quantifying the amount of TR-2 in said second sample;
e) comparing the amount of TR-2 present in the first and second samples; a difference in the amount of TR-2 present in said first and second samples being indicative of disease progression or regression.

15-19. (canceled)

20. An immunogenic composition comprising an effective amount of an isolated protein fragment having an amino acid sequence that corresponds to a portion of the extracellular domain of TR-2.

21. The immunogenic composition of claim 20, wherein the isolated protein fragment has an amino acid sequence corresponding to SEQ ID No. 1.

22. A vaccine effective in treating Alzheimer's disease in a human subject comprising an isolated protein fragment having the amino acid sequence set forth in SEQ ID NO:1 and a pharmaceutically acceptable carrier.

23-26. (canceled)

27. A method of treating Alzheimer's disease comprising
administering to a human patient an inhibitor compound that prevents TR-2 release by inhibiting the enzyme(s) that cleave TR-2 from the cell surface;
administering to said patient a compound that prevents cleaved TR-2 from interacting with beta-amyloid; and
using RNA interference technology to prevent the cellular expression of TR-2.

28-30. (canceled)

31. The method of claim 9, wherein the antibody is specific for an epitope of TR-2 having an amino acid sequence or partial amino acid sequence corresponding to SEQ ID No. 1


SEQUENCE LISTING (5 sequences)

SEQ ID NO: 1 - protein, 20 aa, Homo Sapien
DDRENDLFLCDTNTCKFDGE

SEQ ID NO: 2 - DNA, 31 bp, Artificial Sequence
  Synthesized using artificial sequence “tt” placed before theprimers for amplification purposes, followed by artificial DNA sequence “SacI” (gagctc), artificial DNA sequence “Kozak Sequence”(gccacc) followed by sequence obtained from organism Mus Domesticus.
ttgagctcgccaccatggtgctgtgggagtc

SEQ ID NO: 3 - DNA, 32 bp, Artificial Sequence
  Synthesized using artificial sequence “cctt” placed before theprimers for amplification purposes, followed by artificial DNA sequence “Not1” (gcggccgc) followed by sequence (ctagattaaccgtgtggatg) obtained from mouse organism Mus Domesticus.
ccttgcggccgcctagattaaccgtgtggatg

SEQ ID NO: 4 - DNA, 28 bp, Artificial Sequence
  Synthesized using artificial sequence “tt” placed before theprimers for amplification purposes, followed by artificial DNA sequence “Not1” (gcggccgc) followed by sequence (ccggacagggccaggaac) obtained from mouse organism Mus Domesticus.
ttgcggccgcccggacagggccaggaac

SEQ ID NO: 5 - DNA, 21 bp, Homo Sapien
cccgggagacggcggcggtgg